(19) United States
(12) Patent Application Publication
(10) Pub. No.: US 2007/0270720 A1
(43) Pub. Date: Nov. 22, 2007

(54) Noninvasive physiologic pressure measurement

(76) Inventor: William R. Fry, Colorado Springs, CO (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/429,014

(22) Filed: May 4, 2006

(51) Int. Cl. A61B 5/103

(52) U.S. Cl. 600/587

(57) ABSTRACT

An instrument for at least estimating physiologic pressure such as central venous pressure (CVP) non-invasively includes an ultrasound probe coupled to a display that shows the deformation of a vein as pressure is applied outside the body through the skin and soft tissues. A pressure transducer is applied to the skin to compress a vein, and an indicator coupled to the transducer shows the amount of pressure being applied when the ultrasound probe shows that the vein has collapsed. In the preferred embodiment, the transducer is an inelastic balloon filled with a liquid such as water, and the indicator is in fluid communication with the balloon. The indicator may be a mechanical gauge or meter, or may include a material that converts applied pressure to an electrical signal, in which case the electrical signal may be interfaced to a numerical readout. At least the probe and the transducer are preferably disposed in a hand-held housing.

### FIELD OF THE INVENTION

This invention relates generally to blood pressure monitoring and, in particular, to a noninvasive method and apparatus for measuring central venous pressure (CVP).

### BACKGROUND OF THE INVENTION

Blood pressure in the vena cava just outside the right atrium is called central venous pressure (CVP). Under normal circumstances an increased venous return results in an augmented cardiac output, without significant changes in venous pressure. However, with poor right ventricular function, or an obstructed pulmonary circulation, the right atrial pressure rises. Loss of blood volume or widespread vasodilation will result in reduced venous return and a fall in right atrial pressure and CVP.

CVP is often used to make estimates of circulatory function, in particular cardiac function and blood volume. While CVP does not measure either of these directly, taken in the context of the other physical signs useful information can be gained. The supply of blood to the systemic circulation is controlled by the left ventricle. In a normal patient the CVP closely resembles the left atrial pressure and is usually used to predict it. However, in patients with cardiac disease the right and left ventricles may function differently.

Central venous access currently involves the placement of a venous catheter in a vein that leads directly to the heart. CVP may then be measured using a manometer filled with intravenous fluid attached to the central venous catheter and ‘zeroed’ at the level of the right atrium. An alternative to the manometer involves the use of a butterfly needle inserted into the rubber injection port of ordinary intravenous tubing. In Intensive Care Units, electronic transducers may be connected which give a continuous readout of CVP along with a display of the waveform.

Since standard CVP measurement practice is an invasive, relatively time-consuming process, noninvasive techniques have been investigated. A device which enables a non-invasive estimation of central venous pressure is described in U.S. Pat. No. 5,788,641. The device is comprised of a substantially straight bar having a bottom portion and a top portion. A graduated scale is affixed to or etched into the surface of the bar beginning approximately at the bottom portion. A pointer comprising a pivoting base, a telescoping member and a distal end is movably attached to the bar at its pivoting end such that the pointer slides along the length of the bar. The pointer further comprises a bubble-type indicator means formed integrally therewith. The distal end of the pointer extends in a straight manner from the bar and is extended by the telescoping member. The pointer is rotatable with respect to the bar such that the angle between the bar and the pointer is adjustable. Detents in the pivoting base fix and hold the pointer at several preset angles.

To use the device, a physician first places the pointer at an angle, typically 45 degrees with respect to the bar, and the bar is placed parallel to the frame of a bed. The head of the bed is adjusted until the bubble-type indicating means indicates that the pointer is horizontal and the head of the bed is elevated at a 45 degree angle. Next, the pointer is placed at a 90 degree angle with respect to the bar by engaging the detent at the 90 degree recess and the bottom portion of the bar is positioned on the sternal angle such that the bubble-type indicating means indicates that the pointer is horizontal and the bar is vertical.

This is the critical step in the procedure in that an accurate estimate of the height of visible pulsations is based on the bar being in a vertical orientation and the pointer being in a horizontal orientation. The bubble-type indicating means enables the user to fix the pointer along a true horizontal and the 90 degree detent rigidly holds the pointer at a 90 degree angle with respect to the bar.

The telescoping distal end of the pointer is then extended to a position close to the patient's neck and the height of the pointer is adjusted by sliding the pivoting base along the length of the graduated bar. The distance between the sternal angle and the height of highest visible pulsations in the internal jugular vein is then gauged according to the graduated scale.

More automated, electronic approaches have also been described. In U.S. Pat. No. 4,986,277, a method and apparatus for measuring CVP is disclosed, along with a transducer for measuring CVP in infants. Changes in CVP may also be monitored by employing the method and apparatus of the invention. According to the invention, a transducer is disposed on the neck of a subject (or on the head in the case of infants), and the signal from the transducer is processed to obtain the cardiac component. Thereafter, the vertical distance from the transducer to a reference level is adjusted until the position is located at which the signal transitions between a venous configuration and an arterial or mixed venous-arterial configuration, at which position the vertical distance approximates CVP.

In view of the existing art, the need remains for a simple yet effective, noninvasive approach to CVP measurement.

### SUMMARY OF THE INVENTION

This invention is directed to instrumentation for non-invasive physiologic pressure measurement. In addition to central venous pressure (CVP), the invention is applicable to physiologic pressure measurement with ultrasound guidance to estimate intraocular pressure, arterial pressure, intra-abdominal pressure, and extremity compartment pressure. The pressure measurement is ultrasound guided and could be done hydraulically, pneumatically, or via piezoelectric effect.

The preferred embodiment includes an ultrasound probe coupled to a display that shows deformation in response to applied pressure. In the case of CVP, a pressure transducer is applied to the skin to compress a vein, and an indicator coupled to the transducer shows the amount of pressure being applied when the ultrasound probe shows that the vein has collapsed or become compressed to a predetermined level. Although other superficial veins may be used, including the cephalic, median cubital, basilic, and saphenous, the external jugular is preferred for its location, size, and ability to provide an estimate of CVP.

In the preferred embodiment, the transducer is an inelastic balloon filled with a liquid such as water, and the indicator is in fluid communication with the balloon. The indicator may be a mechanical gauge or meter, or may include a material that converts applied pressure to an electrical signal, in which case the electrical signal may be interfaced to a numerical readout. At least the probe and the transducer are preferably disposed in a hand-held housing.

### BRIEF DESCRIPTION OF THE DRAWINGS

FIG. 1A is a drawing that demonstrates the invention before the application of pressure;

FIG. 1A is a drawing that demonstrates the invention following the application of pressure;

FIG. 2 shows an alternative embodiment of the invention utilizing an electronic pressure sensor; and

FIG. 3 shows another alternative embodiment of the invention wherein pressure is displayed on the same monitor used to show the ultrasonic imagery.

### DETAILED DESCRIPTION OF THE INVENTION

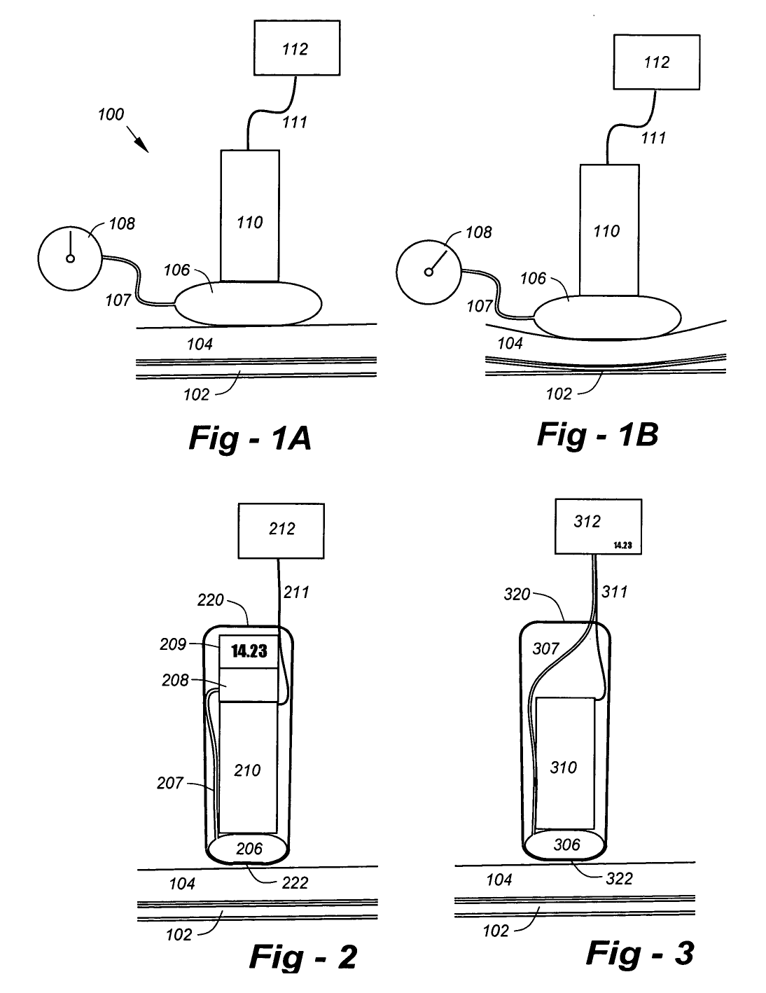

Reference is now made to the drawings, wherein FIG. 1A illustrates at 100 a preferred embodiment of the invention prior to application to the skin of a subject. The apparatus includes an ultrasound probe 110 connected to a display 112 through cable 111. Hand-held probes of this kind are available and known to those in the art.

The probe 110 has a distal end which, in this case, is supported against a balloon or bladder 106. The device 106, preferably flexible but inelastic, is in fluid communication through conduit 107 to a readout device 108. Although an analog/mechanical dial-type gauge or meter is shown, a component such as a piezoelectric crystal may be used to convert pressure into an electrical signal to drive a digital display, as shown in FIG. 2, for example. Although the detailed description focuses on central venous pressure (CVP), the invention is applicable to physiologic pressure measurement with ultrasound guidance to estimate intraocular pressure, arterial pressure, intra-abdominal pressure, and extremity compartment pressure.

The pressure measurement is ultrasound guided and could be done hydraulically, pneumatically, or via piezoelectric effect. Various materials may be used for the device 106, so long as it allows the probe 110 to image the vein 102 through the skin and soft tissues 104. Using the apparatus of FIG. 1A, the probe is urged toward the subject while viewing display 112. At the point when the vein 102 collapses, as shown in FIG. 1B, the readout device 108 indicates the level of pressure needed to counteract venous pressure which, in turn is indicative of CVP.

To ensure a more accurate reading, the readout 108 may be calibrated to CVP in advance of use. If an electrical transducer and readout are used, a look-up table or other calibration circuitry may be used. Although in the preferred embodiment the probe 110 and device 106 are shown aligned on a common axis, it may be possible to place them side-to-side if sufficiently close to one another. The device 106 may be filled with a gas or liquid, though a liquid such as water is preferred as liquids an incompressible.

FIG. 2 shows an alternative embodiment of the invention incorporating a balloon or bladder 206 coupled to an electrical pressure transducer 208 driving a digital readout 209. FIG. 2 also shows the use of a hand-held housing 220 which may be used with any of the disclosed embodiments. The housing 220 includes a deformable end 222 for more conformal skin/tissue contact and accurate results.

Although FIG. 2 shows a housing 220 containing the probe 210, transducer and readout, the transducer and/or display may be positioned at the location of the ultrasound display 312 as shown in FIG. 3. In this case housing 320 includes an inelastic balloon or bladder 306 disposed behind a deformable tip 322 in fluid communication through line 307 with a pressure transducer (not shown) disposed in display 312. Alternatively, an electrical pressure transducer such as 2087 may be used with an electrical connection within cable 311, to provide a readout on display 312. Such arrangements allow a practitioner to view only on location while applying the probe.

I claim:

1. An instrument for estimating physiologic pressure non-invasively, comprising:
an ultrasound probe coupled to a display that shows the deformation of a body organ or vessel as pressure is applied thereto;
a pressure transducer configured to apply pressure to the organ or vessel; and
an indicator coupled to the transducer showing the amount of pressure being applied when the ultrasound probe shows that the organ or vessel has collapsed or become compressed to a predetermined level.

2. The instrument of claim 1, wherein:
the transducer is an inelastic balloon filled with a gas or liquid; and
the indicator is in fluid communication with the balloon.

3. The instrument of claim 2, wherein the indicator is a mechanical gauge or meter.

4. The instrument of claim 2, wherein:
the indicator includes a material that converts applied pressure to an electrical signal; and
the electrical signal is interfaced to a numerical readout.

5. The instrument of claim 1, wherein:
the transducer is composed of a material that converts applied pressure to an electrical signal; and
the electrical signal is interfaced to a numerical readout.

6. The instrument of claim 1, wherein at least the probe and the transducer are disposed in a hand-held housing.

7. The instrument of claim 1, wherein:
the ultrasound probe has a distal end; and
the pressure transducer is supported between the distal end of the probe and the skin.

8. A method of non-invasively estimating central venous pressure (CVP), comprising the steps of:
applying pressure to a vein outside the body through the skin;
monitoring the compression of the vein using ultrasound; and
using the pressure at the point when the vein collapses as an estimate of central venous pressure.
